(19) United States
(12) Patent Application Publication
(10) Pub. No.: US 2013/0344125 A1
(43) Pub. Date: Dec. 26, 2013

(54) DRUG DELIVERY DEVICE

(71) Applicants: Thiresen Govender, Johannesburg (ZA); Viness Pillay, Johannesburg (ZA); Yahya Essop Choonara, Johannesburg (ZA); Lisa Claire Du Toit, Johannesburg (ZA); Girish Modi, Johannesburg (ZA); Dinesh Naidoo, Johannesburg (ZA); Maluta Steven Mufamadi, Johannesburg (ZA)

(72) Inventors: Thiresen Govender, Johannesburg (ZA); Viness Pillay, Johannesburg (ZA); Yahya Essop Choonara, Johannesburg (ZA); Lisa Claire Du Toit, Johannesburg (ZA); Girish Modi, Johannesburg (ZA); Dinesh Naidoo, Johannesburg (ZA); Maluta Steven Mufamadi, Johannesburg (ZA)

(21) Appl. No.: 13/989,402

(22) PCT Filed: Nov. 28, 2011
(86) PCT No.: PCT/IB2011/055345

§ 371 Date: Sep. 9, 2013

(30) Foreign Application Priority Data
Nov. 26, 2010 (ZA) 2010/03743
Nov. 26, 2010 (ZA) 2010/07276

(51) Int. Cl. A61K 9/00; A61K 31/5415

(52) U.S. Cl. CPC A61K 9/0085; A61K 31/5415
USPC 424/423; 514/226.2

(57) ABSTRACT

The invention provides an implantable intracranial device for the site-specific delivery of a pharmaceutically active agent to a human or animal for treating a mental or neurological disorder, such as Alzheimer's disease, schizophrenia or other psychoses. The biodegradable device includes a pharmaceutically active agent for treating the disorder, polymeric nano-lipoparticles into or onto which the pharmaceutically active agent is embedded; and a polymeric matrix or scaffold incorporating the nano-lipoparticles. The nano-lipoparticles can be in the form of nano-liposhells or nano-lipobubbles. The nano-liposhells or nano-lipobubbles can include an essential fatty acid or can be conjugated to a peptide ligand which targets the device to a specific cell into which the therapeutic agent can be delivered. The device can be implanted in the sub-arachnoid space in the region of the frontal lobe of the brain.

### FIELD OF THE INVENTION

The present invention relates to biodegradable drug delivery devices which are implantable in the cranium for delivering pharmaceutically active agents to the brain, and in particular, for treating Alzheimer's disease and psychotic disorders such as schizophrenia.

### BACKGROUND TO THE INVENTION

One of the challenges in treating most neurological or mental disorders is the difficulty in delivering therapeutic agents to the brain. Many potentially important diagnostic and therapeutic agents or genes are unable to cross the blood-brain barrier (BBB) or do not cross the BBB in adequate amounts.

Mechanisms for drug targeting in the brain involve going either “through” or “behind” the BBB. Modalities for drug delivery through the BBB entail its disruption by osmotic means; biochemically by the use of vasoactive substances such as bradykinin; or even by localized exposure to high-intensity focused ultrasound (HIFU). Other methods used to get through the BBB entail the use of endogenous transport systems, including carrier-mediated transporters such as glucose and amino acid carriers; receptor-mediated transcytosis for insulin or transferrin; and the blocking of active efflux transporters such as p-glycoprotein. Methods for drug delivery behind the BBB include intracerebral implantation (such as with needles) and convection-enhanced distribution.

Nanotechnology may also help in the transfer of drugs across the BBB. A significant amount of research in this area has been spent exploring methods of nanoparticle-mediated delivery of antineoplastic drugs to tumors in the central nervous system. For example, radiolabeled polyethylene glycol coated hexadecylcyanoacrylate nanospheres targeted and accumulated in a rat gliosarcoma. Recently, researchers have been trying to build liposomes loaded with nanoparticles to gain access through the BBB. More research, however, is needed to determine which strategies will be most effective and how they can be improved.

Alzheimer's disease and schizophrenia are just two examples of the many mental and neurological disorders (NDs) which are difficult to treat.

Alzheimer's disease (AD) is one of the most common diseases of the Central Nervous System (CNS) marked by decline in memory and cognitive performance, and defects in visual and motor coordination. It is estimated that there are about 26 million people suffering with Alzheimer's disease worldwide. Alzheimer's disease is associated with the progressive accumulation of β-amyloid plaques in the brain during aging and is identified by extracellular neuritic plaques and neurofibrillary tangles. The major component of the β-amyloid plaques is the beta amyloid (Aβ) peptide, which is a cleavage product of the amyloid precursor protein (APP). These Aβ peptides range in size from 37 to 43 amino acids; however, Aβ peptide 40-43 are known to act as a pathogenic seed for Aβ aggregation and amyloid plaque formation because they are more hydrophobic compared to the shorter amyloid peptides. There is considerable evidence that Aβ peptide has to undergo a process of polymerisation in order to produce neurotoxic forms of amyloid. A study has shown that oxidative stress, inflammation, and free radicals may be the primary cause of Alzheimer's disease neurotoxicity.

Ddonepezil, rivastigmine and galantamine are the drugs currently used to treat Alzheimer's disease. Ddonepezil and galantamine are able to inhibit acetylcholinesterase whereas rivastigmine inhibits the enzyme butyrylcholinesterase. Supplementary agents, antioxidants such as vitamins C, Vitamin E, and beta-carotene can also be considered as anti aging therapy that provide protection against oxidative damage to Alzheimer's disease patients.

One of the current challenges for the effective treatment of neurological disorders, including Alzheimer's disease is the need to bridge the gap between the indispensable drug therapies that are available and the improvement in the mode of drug delivery to ensure minimal drug toxicity, improved efficacy and a superior quality of life for patients challenged with NDs. The treatment of Alzheimer's disease following systemic drug administration is still challenging due to the existence of the highly restrictive Blood-Brain Barrier (BBB) as discussed above. It has been suggested that the BBB restricts the entry of substances entering the brain based on particle size and endothelial permeability. The BBB comprises tight cell junctions and ATP-dependent efflux pumps that restricts the delivery of drug molecules into the brain, thus making the therapy of Alzheimer's disease via the systemic route significantly difficult. Although lipophilic molecules, peptides, nutrients and polymers may satisfy penetrability requirements, these molecules are associated with the inability to access and penetrate targeted regions within the brain, or are inherently non-specifically taken up by sensitive normal tissues and cells.

The vast prevalence of schizophrenia, its chronic and debilitating nature, and the risk of relapse and suicide makes effective treatment for the disorder mandatory. Apart from the problems associated with getting therapeutic agents transferred across the BBB as discussed above, the success of maintenance therapy in schizophrenia also depends on a number of variables, including the constant release of neurotherapeutics, a reduction in the dosing frequency, a greater antipsychotic drug bioavailability and ultimately improved patient compliance, many of which are not achievable by conventional oral formulation schizophrenia therapy (Pranzatelli, 1999; Cheng et al., 2000). Currently, the convenient and preferred drug delivery system for antipsychotic drugs includes conventional tablet or capsule formulations. As with most conventional oral drug delivery systems, they exhibit first-order drug release kinetics where drug levels are higher after ingestion but decrease exponentially, not allowing optimum prolonged plasma levels for therapeutic efficacy, resulting in dose-dependant side effects.

An impediment in the long-term treatment and positive therapeutic outcome in schizophrenia is non-compliance with treatment regimes, which may be as a result of numerous factors. One of the most significant factors is the intolerable side-effects associated with anti-psychotic medication. Atypical antipsychotic drugs are the popular choice in the treatment of schizophrenia due to their lack of associated extrapyramidal side-effects and their superior safety profile regarding prolactin. Examples of atypical antipsychotics include olanzapine, which has been linked to weight gain and an increased lipid and glucose metabolism. Clozapine, another atypical antipsychotic, causes agranulocytosis and has been implicated in cases of fatal constipation. It has been documented that the use of antipsychotics in general increases the risk of metabolic syndrome. However, due to the severity and complexity of schizophrenia, physicians continue to prescribe antipsychotic drugs despite their life-threatening ramifications. Non-compliance is also related to the dosing frequency of certain antipsychotic medication. For example, oral treatment for schizophrenia may be dosed from two to four times a day.

Several clinical studies have proved the safety and efficacy of long-acting injectable depot formulations of atypical antipsychotic drugs However, there are limitations of depot formulations that may affect compliance or efficacy. Disadvantages of depot formulations include patients' unwillingness to receive injections, the inability to rapidly cease the medication if serious side-effects occur, difficulties in dose adjustments, complex and intricate pharmacokinetics, abscess formation, pruritis and extended periods of pain at the injection site. Furthermore, depot formulations containing the decanoate functional group are restricted by their chemistry (Kane, et al., 1998; McCauley and Connolly, 2004; Rabin, et al., 2008).

There is therefore a need for new methods or compositions which can at least partially overcome the difficulty of delivering therapeutic agents to specific regions of the brain so as to treat mental or neurological disorders.

### SUMMARY OF THE INVENTION

According to a first embodiment of the invention, there is provided an implantable intracranial device for the delivery of a pharmaceutically active agent to a human or animal for treating a mental or neurological disorder, the device comprising:
a pharmaceutically active agent for treating the disorder;
polymeric nanoparticles into or onto which the pharmaceutically active agent is embedded; and
a polymeric matrix incorporating the nanoparticles.

The mental or neurological disorder may be Alzheimer's disease or a psychotic disorder such as schizophrenia.

The pharmaceutically active agent may be a cholinesterase inhibitor such as donepezil hydrochloride, rivastigmine or galantamine; an NMDA receptor antagonist such as memantine; an atypical antipsychotic such as amisulpride, aripiprazole, asenapine, bifeprunox, blonanserin, clotiapine, clozapine, iloperidone, lurasidone, mosapramine, olanzapine, paliperidone, perospirone, pimavanserin, quetiapine, remoxipride, risperidone, sertindole, sulpiride, vabicaserin, ziprasidone or zotepine; or a typical antipsychotic such as chlorpromazine, thioridazine, mesoridazine, levomepromazine, loxapine, molindone, perphenazine, thiothixene, trifluoperazine, haloperidol, fluphenazine, droperidol, zuclopenthixol or prochlorperazine, or a salt thereof.

The nanoparticles may be nano-lipoparticles, and are preferably nano-liposhells or nano-lipobubbles.

The nano-lipoparticles may be formed from a composition comprising a polymer and the pharmaceutically active agent, and the composition may additionally comprise at least one phospholipid and/or essential fatty acid (such as omega 3 fatty acid). For example, the nano-lipoparticles may be formed from a composition comprising polycaprolactone, the pharmaceutically active agent and omega 3 fatty acid, or may be formed from a composition comprising 1,2-distearoyl-sn-glycero-phosphatidylcholine (DSPC); cholesterol; 1,2-distearoyl-sn-glycero-3-phosphatidylcholinemethoxy(polyethyleneglycol)-2000] (DSPE-m PEG2000) conjugate and the pharmaceutically active agent.

The nano-lipoparticles may additionally comprise a peptide ligand for targeting the nano-lipoparticles to a target molecule. The peptide ligand may be conjugated to the nanoparticles, and is preferably capable of binding to the serpin-enzyme receptor complex (SEC receptor) in the brain. The peptide ligand may comprise an amino acid sequence selected from the group consisting of KVLFLM (SEQ ID NO: 1), KVLFLS (SEQ ID NO: 2) and KVLFLV (SEQ ID NO: 3).

The polymeric matrix may be formed from a composition comprising ethylcellulose and modified polyamide 6, 10, or from a composition comprising chitosan, eudragit and sodium alginate.

The polymeric matrix may be porous.

The device may be implantable in the sub-arachnoid space of a human or animal.

The device may be biodegradable.

In a preferred embodiment:
the pharmaceutically active agent may be an agent for treating Alzheimer's disease;
the polymeric nanoparticles may be nano-lipobubbles formed from 1,2-distearoyl-sn-glycero-phosphatidylcholine (DSPC); cholesterol; 1,2-distearoyl-sn-glycero-3-phosphatidylcholinemethoxy(polyethyleneglycol)-2000] (DSPE-mPEG2000) conjugate and the pharmaceutically active agent and may be coupled to a peptide ligand having an amino acid sequence of KVLFLM (SEQ ID NO: 1), KVLFLS (SEQ ID NO: 2) or KVLFLV (SEQ ID NO: 3) targeting the serpin-enzyme complex receptor (SEC receptor) in the brain; and
the polymeric matrix may be a porous scaffold formed from chitosan, eudragit and sodium alginate.

In an alternative preferred embodiment:
the pharmaceutically active agent may be an antipsychotic agent for treating schizophrenia;
the polymeric nanoparticles may be nano-liposhells formed from polycaprolactone, the pharmaceutically active agent and omega 3 fatty acids; and
the polymeric matrix may be formed from ethylcellulose and modified polyamide 6, 10.

According to a second embodiment of the invention, there is provided a method of manufacturing an implantable intracranial device substantially as described above, the method comprising the steps of:
forming nano-lipoparticles containing a pharmaceutically active agent; and
incorporating the nano-lipoparticles into a polymeric matrix.

According to a third embodiment of the invention, there is provided a method of treating a mental or neurological disorder comprising implanting a device substantially as described above into the cranium of a patient.

### BRIEF DESCRIPTION OF THE FIGURES

FIG. 1 shows the characterization of targeted nano-lipobubbles (NLBs) of one embodiment of the invention in terms of size distribution and Zeta potential. (A) a size distribution profile of non-targeted nano-lipobubbles; (B) synthetic peptide ligand only; (C) targeted nano-lipobubbles; and (D) an overall zeta potential distribution profile of targeted nano-lipobubbles.

FIG. 2 shows FTIR spectra of targeted nano-liposomes (NLPs) with synthetic peptide ligand (KVLFLM (SEQ ID NO: 1).

FIG. 3 shows FTIR spectra of targeted nano-liposomes (NLPs) with synthetic peptide ligand (KVLFLS (SEQ ID NO: 2).

FIG. 4 shows DSC thermograms of the DSPC, Cholesterol, DSPE-mPEG, non-targeted nano-lipobubbles and targeted nano-lipobubbles with synthetic peptide ligand (KVLFLS) (SEQ ID NO: 2).

FIG. 5 shows the cytotoxic activity of synthetic peptide targeted nano-lipobubbles, non-targeted nano-lipobubbles and nano-liposomes.

FIG. 6 shows fluorescence profiles of rhodamine-labelled non-targeted nano-lipobubbles and targeted nano-lipobubbles.

FIG. 7 shows scanning electron microscopy of the surface of chitosan/Eudragit RS-PO/sodium alginate polymer matrix scaffold at different magnifications (x1360 and x2760).

FIG. 8 shows confocal fluorescence micrographs of rhodamine-labeled targeted nano-lipobubbles inside porous polymer matrix scaffold: (A) Porous scaffold only; and (B) rhodamine-labeled targeted nano-lipobubbles distribution inside the porous scaffold.

FIG. 9 shows the relative size of the polymeric implantable device of the present invention.

FIG. 10 shows force-distance profiles at the centre of the polymeric device of Example 2.

FIG. 11 shows FTIR spectra of the polyamide 6, 10, ethylcellulose and polyamide-ethylcellulose device of Example 2 synthesized by modified immersion precipitation reaction.

FIG. 12 shows SEM images of the polymeric devices of Example 2 at varying magnifications.

FIG. 13 shows a typical intensity profile obtained showing a size distribution profile of the chlorpromazine-loaded nano-liposhells of Example 2.

### DETAILED DESCRIPTION OF THE INVENTION

An implantable intracranial device for the site-specific delivery of a pharmaceutically active agent to a human or animal for treating a mental or neurological disorder is described. The biodegradable device (or dosage form) includes a pharmaceutically active agent for treating the disorder, polymeric nanoparticles into or onto which the pharmaceutically active agent is embedded; and a polymeric matrix or scaffold incorporating the nanoparticles. The device can be implanted in the sub-arachnoid space in the region of the frontal lobe of the brain.

The mental or neurological disorder is typically a degenerative neurological disorder such as Alzheimer's disease or schizophrenia or other psychoses. As chronic treatment is required for these disorders, the device is able to release the pharmaceutically active agent in a controlled and sustained manner for extended and prolonged periods of time.

Pharmaceutically active agents for treating Alzheimer's disease include cholinesterase inhibitors such as donepezil hydrochloride, rivastigmine or galantamine and NMDA receptor antagonists such as memantine.

Pharmaceutically active agents for treating schizophrenia include atypical antipsychotics such as amisulpride, aripiprazole, asenapine, bifeprunox, blonanserin, clotiapine, clozapine, iloperidone, lurasidone, mosapramine, olanzapine, paliperidone, perospirone, pimavanserin, quetiapine, remoxipride, risperidone, sertindole, sulpiride, vabicaserin, ziprasidone or zotepine; and typical antipsychotics such as chlorpromazine, thioridazine, mesoridazine, levomepromazine, loxapine, molindone, perphenazine, thiothixene, trifluoperazine, haloperidol, fluphenazine, droperidol, zuclopenthixol or prochlorperazine.

The nanoparticles can be nano-lipoparticles, and are typically nano-liposhells or nano-lipobubbles.

The nano-lipoparticles can be formed from a composition comprising a biodegradable polymer and the pharmaceutically active agent, and the composition can additionally comprise at least one phospholipid and/or essential fatty acid (such as an omega 3 fatty acid). In one embodiment, the nano-lipoparticles are formed from a composition comprising polycaprolactone, the pharmaceutically active agent and an omega 3 fatty acid. In another embodiment, the nano-lipoparticles are formed from a composition comprising 1,2-distearoyl-sn-glycero-phosphatidylcholine (DSPC); cholesterol; 1,2-distearoyl-sn-glycero-3-phosphatidylcholinemethoxy(polyethyleneglycol)-2000] (DSPE-mPEG2000) conjugate and the pharmaceutically active agent. They may also include rhodamine-labeled phosphatidylethanolamine (Rh-DSPE). The ratio of DSPC/Chol/DSPE-mPEG 2000 can range from about 50/50/10 to about 75/25/10 mg/mg/mg, and

The nano-liposhells can be formed using a modified melt-dispersion technique and the nano-lipobubbles can be prepared by a reverse phase evaporation technique and nitrogen gas. The nano-liposhells or nano-lipobubbles can have an irregular shape.

The pharmaceutically active agents can be embedded within, encapsulated in or attached to the nano-liposhells or nano-lipobubbles.

The nano-lipoparticles can additionally comprise an affinity moiety such as a peptide ligand for targeting the nano-lipoparticles to a target molecule. The peptide ligand is preferably selected so as to be capable of binding to the serpin-enzyme complex receptor (SEC receptor) which is over-expressed in the brain in Alzheimer patients. Synthetic peptides corresponding to a peptide of 6 amino acids isolated from human apolipoprotein A-1 and having one of the following amino acid sequences are particularly suitable: KVLFLM (SEQ ID NO: 1), KVLFLS (SEQ ID NO: 2) or KVLFLV (SEQ ID NO: 3).

Sequence motifs bearing homology with this pentapeptide domain were found in the Aβ peptide common in AD. SEC-receptor is also shown to mediate internalization of Aβ-peptide in neuronal cell-lines (PC12). Previous documented research demonstrated that polylysine was able to conjugate to a synthetic peptide (soluble) transfer gene into heptoma cell-lines via SEC-receptor. However, research has also demonstrated that the Aβ 25-35 peptide (insoluble) was not recognized at all by SEC-receptors and retained its full toxic/aggregating properties. Further research demonstrated that human apolipoprotein A-I (ApoA-I) sequence motifs bared homology with the Aβ peptide. The previous research also demonstrated binding between ApolA-1 to Aβ peptide and preventing Aβ peptide from inducing neurotoxicity that is commonly found in AD. Therefore developing a novel drug delivery strategy employing the ApoA-I (sequences) as targeted ligands for delivering neuroactive drug to a specific receptor (SEC-receptor) is proposed in this patent.

The peptide ligand can be conjugated or coupled, preferably covalently, to the nanoparticles using N′-dicyclohexylcarbodiimide (DCC) and N-hydroxysulfosuccinimide (NHS) conjugate.

The DSPC/Chol/DSPE-mPEG 2000/peptide ligand in the nanoparticles can be present in a ratio of from about 50/50/10/1 to about 75/25/10/1 mg/mg/mg/mg, and the DSPC/Chol/DSPE-mPEG 2000/Rh-DSPE/peptide ligand in the nanoparticles can be present in a ratio of from about 50/50/10/1/1 to 75/25/10/1/1 mg/mg/mg/mg/mg.

In one embodiment of the invention, the polymeric matrix or scaffold is a membranous polymer formed from a composition comprising biodegradable polymers with low antigenicity, typically ethylcellulose and modified polyamide 6, 10, in a modified immersion precipitation reaction. The ethylcellulose and polyamide 6, 10 can be solubilized with acetone and formic acid 85%, respectively. Double-de-ionized water is used as a non-solvent to precipitate the polymeric blend of ethylcellulose and polyamide 6, 10.

In another embodiment, the polymeric matrix or scaffold is formed from a composition comprising chitosan, eudragit and sodium alginate, typically in a ratio of from 1/1/1 to about 2/1/1 chitosan/sodium alginate/Eudragit RS-PO.

A planar surface of the device can be coated with a substance, preferably a hydrophobic polymer, controlling the nano-liposhells or nano-lipobubbles and subsequent bioactive release from that surface of the pharmaceutically active agent.

De-ionized water molecules can be used as porogen agents to make the polymeric matrix or scaffold porous. The pores are typically <20 microns in size and relatively uniform in shape.

In one embodiment of the invention (described in more detail in Example 1), the device is an implantable dosage form for treating Alzheimer's disease and the pharmaceutically active agent is an Alzheimer's drug which is incorporated into nano-lipobubbles which have a synthetic peptide ligand for specific site-targeting (Forssen and Willis, 1998, Torchilin, 2008). The drugs and perfluorocarbon gas are incorporated into the core of the nano-lipobubbles (Klibanov, 1999; Cavalieri et al., 2006; Hernot and Klibanov, 2008). The nano-lipobubbles are surface coated with PEG and have a nanosize diameter range, a size distribution from 100 nm to 200 nm, and a zeta potential towards a negative charge. The targeting ligands are covalently conjugated or engineered within the surface of the nano-lipobubbles with coupling techniques using crosslinking agents such as NHS and DCC (Nobs et al., 2004).

The polymeric matrix or scaffold of the device is formed from a combination of chitosan/Eudragit/sodium alginate at a ratio of from about 1:1:1 to about 2:1:1 mg/mg/mg. In addition, porogen agents such as deionized water molecules are added in order to generate pores within the scaffold. The pre-labelled targeted nano-lipobubbles are incorporated within the polymeric scaffold using either of two methods. In the first method, the targeted nano-lipobubbles are loaded with polymeric solutions during agitation, then lyophilized. In the second method, pre-encapsulated drug-loaded targeted nano-lipobubbles are embedded within the polymeric scaffold via injection. The polymeric matrix or scaffold device would be able to “intelligently” release the pre-labeled or pre-encapsulated drug-loaded targeted nano-lipobubbles in a passive or actively pre-programmed manner.

The release profile of pre-encapsulated drug-loaded nano-lipobubbles will be monitored in a transgenic mouse model of Alzheimer's disease. The release of the targeted nano-lipobubbles from the scaffold will be monitored in terms of its ability to direct and internalize the targeted nano-lipobubbles into target cells via receptors in which the active pharmaceutical composition will be released.

In another embodiment of the invention (described in more detail in Example 2), the device is a dosage form for treating schizophrenia and is implantable in the sub-arachnoid space in the region of the frontal lobe of the brain. The pharmaceutically active agent is a typical or atypical antipsychotic such as chlorpromazine (a cost-effective antipsychotic drug which has declined in its use due to its significantly low bioavailability). The polymer matrix is a membranous polymer composition formed from an ethylcellulose and modified polyamide 6, 10 blend. Bioactive nano-liposhells housing chlorpromazine hydrochloride are matricized within the membranous composition. The nano-liposhells comprise cod-liver oil B.P. and chlorpromazine hydrochloride, encapsulated within a polycaprolactone nano-shell.

Site-specific drug delivery and nano-liposhell technology provided by the delivery device avoid at least some of the difficulties posed by the blood brain barrier (BBB). In addition, the need to administer potentially toxic doses of antipsychotic drugs for the sake of achieving therapeutic doses in the central nervous system is avoided. Furthermore, the nano-liposhells do not have lengthy distances to travel as when injected into the systemic circulation, where they have a greater chance of degrading or breaking down. The active drug can be released from the nano-liposhells by simple diffusion, erosion of the nano-liposhell or evaporation of the core.

The proposed device could result in a decrease in systemic side-effects, a decrease in serum protein binding, reduced hepatic metabolism and peripheral drug inactivation, and polymeric protection of active drug by the membranous scaffold and nano-encapsulation techniques, thereby reducing degradation. Furthermore, large doses of drug can be localized in the areas of the brain where it is required the most. The fact that the device is biodegradable ensures that no additional surgery will be required to remove the device. In addition, chlorpromazine could be released with near zero-order for up to a year in a controlled manner, maintaining optimum levels in the CNS compartment and preventing relapse. It is envisaged that the device of the present invention could improve the bioavailability of the chlorpromazine as a result of site-specific drug delivery, making it a drug of choice for treating schizophrenia once again. In addition, the device combines the benefits of a scheduled drug with a complimentary medicine. Omega 3 fatty acids such as eicosapentaenoic acid (EPA) and docosahexaenoic acid (DHA) and have been shown to have neuroprotective properties in the CNS and could be particularly beneficial to patients with schizophrenia. Cod-liver oil B.P. is rich in both EPA and DHA.

The invention will now be described by way of the following non-limiting examples.

### EXAMPLES

### Example 1

### A Drug Delivery Device for Treating Alzheimer's Disease

### Materials and Methods

### Materials

Phospholipids such as distearoyl-sn-glycero-phosphatidylcholine (DSPC), cholesterol and 1,2-distearoyl-sn-glycero-3-phosphatidyl-ethanolamine-methoxypolyethyleneglycol conjugate (DSPE-mPEG 2000), and rhodamine-labeled phosphatidylethanolamine (Rh-DSPE), chitosan (medium grade molecular weight), Eudrogit RS-PO, sodium alginate, acetic acid glacial were all purchased from Sigma-Aldrich (St. Louis, Mo., USA). N,N′-dicyclohexylcarbodiimide (DCC), N-hydroxysulfosuccinimide, sodium hydroxide (NaOH) and potassium dihydrogen phosphate (KH₂PO₄) were purchased from Saarchem (Pty) Ltd (Brakpan, South Africa). 0.22 μm membrane filters were purchased from Millipore (Billerica, Mass., USA). Nitrogen gas was purchased from Afrox Ltd (Industria West, Germiston, SA). All of the peptide ligands were synthesized by SBS Genetech CO., Ltd (Shanghai, China). The CytoTox-Glo™ Cytotoxicity Assay (Kit) which measure cell viability was purchased from Promega Corporation (Madison, Wis., USA). All the solvents and reagents were of analytical grade and were used as purchased.

### Preparation of the Nanolipobubbles

Nano-lipobubbles were prepared using an adapted reverse phase evaporation technique (Suzuki et al., 2007). DSPC, CHOL and DSPE-mPEG conjugate were dissolved in an organic solvent phase of chloroform/methanol (9:1). Phosphate buffered saline (PBS) (pH 7.4) was added into the lipid solution. Thereafter, the mixture was blended with a probe sonicator (60 rpm, 30 seconds) followed by solvent evaporation using a rotary evaporator on a water-bath with temperature maintained at 65° C. for 2-3 hours. The lipid films which formed were suspended in a round bottom glass tube in 4 mL PBS buffer at pH 7.4. Uni-lamellar liposomes (NLPs) were obtained by freeze and thawing techniques. Liposome solutions were frozen at −70° C. and then re-thawed on a water-bath at 37° C. (repeated 6 times) (Yagi et al., 2000). Size distribution was obtained by gradually extruding through a 0.22 μm pore size polycarbonate membrane filter (Verma et al., 2003, Zhua et al., 2007). The samples obtained were allowed to stabilize for 24 hours at 4° C. 15 mL tubes containing 5 mL of the stabilized nanoliposomes were exposed to nitrogen gas, capped and then placed in a bath type sonicator for 5 minutes in order to form nano-lipobubbles (NLBs).

Covalent Coupling of the Synthetic Peptide Ligands onto the Nano-Lipobubble Surface

Peptide-PEG-nano-lipobubbles were prepared (Yagi et al., 2000; Janssen et al., 2003). In briefly, Nano-lipobubbles with DSPE-mPEG-COOH conjugate were first activated with NHS and DCC) solutions at room temperature for 4 hours. Then, an appropriate quantity of synthetic peptides (KVLFLM-NH2 (SEQ ID NO: 1), KVLFLS-NH2 (SEQ ID NO: 2) or KVLFLT-NH2 (SEQ ID NO: 3)) was added into treated nano-lipobubbles at a ratio of 75/1 mg. The conjugation reaction was stirred overnight at room temperature. The solvents were then precipitated by rotary evaporation on a water-bath with temperature maintained at 65° C. for 2-3 hours. The solutions were then dialyzed against PBS using SnakeSkin™ Pleated Dialysis Tubing (10,000 MWCO; Sigma-Aldrich) for 24 hours to remove unconjugated synthetic peptide ligands. Thereafter, the targeted nano-lipobubbles were stabilized by freeze and thawing techniques. Size distribution was obtained by gradually extruding through a 0.22 μm pore size polycarbonate membrane filter. Targeted nano-lipobubbles were then stored in 4° C. until further use.

### Assessment of the Physicochemical Properties of the Targeted Nano-Lipobubbles

### Determination of Particle Size and Size Distribution

Particle size and size distribution of synthetic peptide ligands, non-targeted nano-lipobubbles and targeted nano-lipobubbles were analyzed by a Zetasizer NanoZS instrument (Malvern Instruments (Pty) Ltd., Worcestershire, UK) at 25° C. Samples were suspended in deionized water, and then extruded through a 0.22 μm pore size polycarbonate membrane filter prior to analysis. Each analysis was performed in triplicate.

### Determination of the Zeta-Potential

Zeta-potential of the targeted Nano-lipobubbles was analyzed by a Zetasizer NanoZS instrument (Malvern Instruments (Pty) Ltd., Worcestershire, UK) at 25° C. Sample was suspended in deionized water, and then extruded through a 0.22 μm pore size polycarbonate membranes filter prior to analysis (carried out in triplicate).

### Fourier Transmission Infrared Spectroscopy Analysis

Fourier Transmission Infrared (FTIR) spectrophotometry of the targeted nano-lipobubbles was performed in order to characterize the potential interactions of the synthetic peptide ligand on the nano-lipobubbles' surface conjugate. The samples were analyzed at high resolution with wavenumbers ranging from 4000 to 400 cm⁻¹ on a Nicolet Impact 400D FTIR Spectrophotometer coupled with Omnic FTIR research grade software (Nicolet Instrument Corp., Madison, Wis., USA).

### Differential Scanning Calorimetry Analysis

DSC experiments were performed by a Mettler Toledo DSC system (DSC-823, Mettler Toledo, Switzerland). A Mettler Stare software system, version 9.x, was used for data acquisition and indium was used to calibrate the instrument. The samples (mg) were transferred into DSC standard aluminum pans and sealed. The samples were analyzed by heating over the temperature range from 0-250° C. at a rate of 10° C./min under an 8 kPa nitrogen atmosphere. Each experiment was repeated three times.

### Neuronal Cell Culture Studies

The PC12 cell-line was used as a model system for primary neuronal differentiation, derived from Rattus norvegicus pheochromocytoma (Greene and Tischler, 1976) and purchased from the Health Science Research Resources Bank (HSRRB, Osaka, Japan). The cells were cultured in RPMI-1640 media (with L glutamine and Sodium Bicarbonate) supplemented with 5% foetal bovine serum, 10% horse serum (both heat inactivated) and 1% penicillin/streptomycin (Sigma-Aldrich) and were maintained in an incubator with humidified atmosphere with 5% CO₂ at 37° C. The cells were cultured or stored in 75 cm tissue culture flasks.

### Cytotoxicity Assay

For the cytotoxicity assay, the PC12 cells were seeded at a density of 10,000 cells per well in flat bottom 96-well plates overnight before the addition of different samples. To assess the cell viability, PC12 cells were first treated with synthetic peptide ligands (KVLFLM (SEQ ID NO: 1) or KVLFLS (SEQ ID NO: 2)) at different concentrations (0.1, 1 and 10 mg/mL), and subsequently with non-targeted nano-lipobubbles and targeted nano-lipobubbles with synthetic peptide ligand (KVLFLM or KVLFLS) at 1 mg/mL concentration. Subsequently, the plates were incubated for 0, and 24 hours at 37° C. in the CO₂ incubator. To determine cytotoxicity at 0 and 24 hour intervals, 50 μL Cytolox-Glo™ Cytotoxicity Assay Reagent was added to each well. The plates were immediately incubated at room temperature for 15 minutes and the dead cell signal measured using a Victor X3 Luminometer PerkinElmer Inc. (Wellesley, Miss., USA). To determined cell viability at 0 and 24 hours, 50 μL of the lysis reagent was added to each well to achieve complete cell lysis. Subsequently, the plate was incubated at room temperature for another 15 minutes and the live cell signal measured by Victor X3 Luminometer (Victor X3, Perkin Elmer, USA). The percentage of viable cells was calculated using the following formula:

$$Cell Viability = 100 \times \frac{\begin{matrix}Average Luminescence for X - \\ Average Luminescence for Blank\end{matrix}}{\begin{matrix}Average Luminescence for Control - \\ Average Luminescence for Blank\end{matrix}}$$

where, Average Luminescence for X % is the luminescence values of cells treated with the various formulations, or peptide ligand or targeted Nano-lipobubbles, Average Luminescence for Blank (i.e. the luminescence of 50 μL substrate which was added to 100 μL medium in an empty well without cells) and A control is the luminescence of untreated cells incubated with the CytoTox-Glo™ CTCA reagent (i.e 50 μL substrate).

### Ex Vivo Uptake of Targeted Nano-Lipobubbles

Targeted Nano-lipobubbles were labelled with fluorescent tags such as rhodamine for ex vivo traceability. PC12 cells at a density 10,000 cells were plated in sterile Nanc 96 well plate (Sepsic, Co, South Africa). On the second day, cells were incubated with rhodamine-labeled targeted nano-lipobubbles and non-targeted nano-lipobubbles or NLPs for 0 hours, 12 hours, and 24 hours at 37° C. The resulting samples were centrifuged at 10,000 g for 20 minutes at 4° C. The aqueous phase was removed and the amount of associated with the rhodamine fluorescent equivalents was measured with a Victor X3 fluorimeter, PerkinElmer Inc. (Wellesley, Miss., USA).

### Fabrication of the Chitosan/Eudragit/Sodium Alginate Porous Scaffold

A Eudragit RS-PO solution was added to a sodium alginate aqueous solution slowly with stirring for 4 hours. An appropriate quantity of the blend was then added to a chitosan solution with stirring for another 24 hours. Deionized water molecules were also added to the chitosan/eudragit/sodium alginate solution at a ratio of 1:10 volume/volume. The blended solutions were poured into a petri dish (diameter, 10 mm; height, 5 mm) and frozen for 48 hours in a −70° C. freezer, followed by lyophilization (Virtis lyophilizer, Virtis®, Gardiner, N.Y.) for 24 hours. The polymeric scaffolds were then analyzed on a scanning electron microscope (SEM), (JEOL, Tokyo, Japan) after samples were first mounted onto metal stubs, using double-sided adhesive carbon tape and then sputter-coated with a thin layer of gold for 90 seconds before generating the photomicrographs.

Labeled Nano-Lipobubbles Encapsulation and Distribution within the Porous Scaffold

The encapsulation and distribution of labeled nano-lipobubbles within the porous scaffold were evaluated after nano-lipobubbles were inserted during agitation. The sample mixtures were refrigerated at −70° C. over 24 hours, and then lyophilized at 25 mTorr (Virtis®, Gardiner, N.Y., USA) for another 24 hours. The encapsulation and distribution studies of the labeled nano-lipobubbles within the interior of the porous scaffold were monitored using confocal microscopy.

In Vitro Release of the Targeted Nano-Lipobubbles from the Porous Scaffold

Samples of the pre-encapsulated rhodamine-labeled targeted nano-lipobubbles within the porous scaffolds were immersed in 20 mL PBS (pH 7.4, 37° C.) and agitated at 20 rpm in a shaking incubator (Labex Stuart SBS40®, Gauteng, and South Africa). Samples were removed for analysis at 0, 10, 20, and 30 days.

### Results and Discussion

### Physicochemical Properties of the Targeted Nano-Lipobubbles

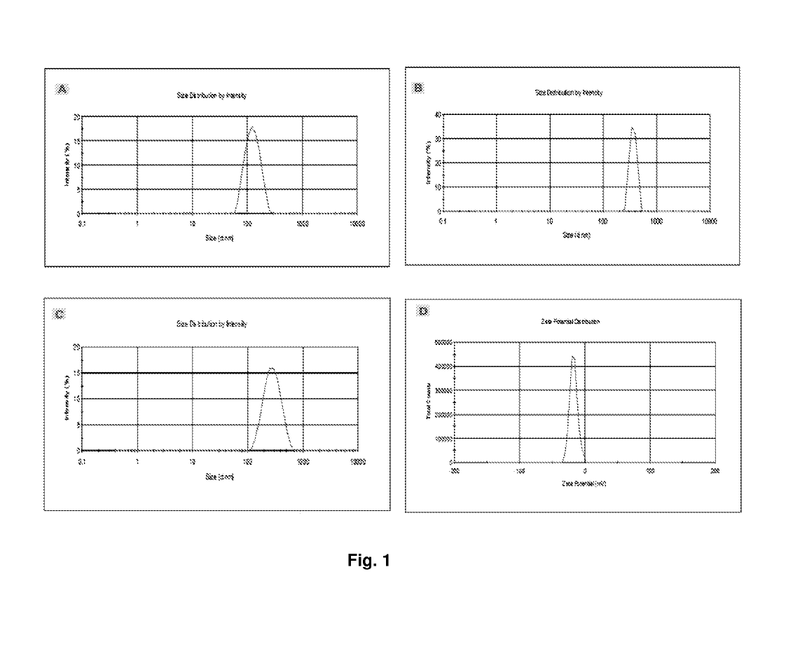

The physicochemical properties of the targeted nano-lipobubbles in terms of size distribution and zeta-potential were examined using the standard method of dynamic light scattering measurement (Zetasizer NanoZS, Malvern Instrument). As shown in FIG. 1, the diameter of the non-targeted nano-lipobubbles was in the range of 129±14 nm. The diameter of synthetic peptide ligand (KVLFLS 9 SEQ ID NO: 2)) alone was in the range of 366±41 nm. When 1 mol % of the linear synthetic peptide ligand incorporated or coupled with circular non-targeted nano-lipobubbles to generate targeted nano-lipobubbles, the size distribution increased from 129 nm to 270 nm when it was compared with non-targeted nano-lipobubbles. This confirms the successfully incorporation of the synthetic peptide ligand on the surface of non-targeted nano-lipobubbles. The overall zeta potential or surface charge of the targeted nano-lipobubbles was −29 mV.

### Assessment of Targeted Nano-Lipobubbles Structural Variations

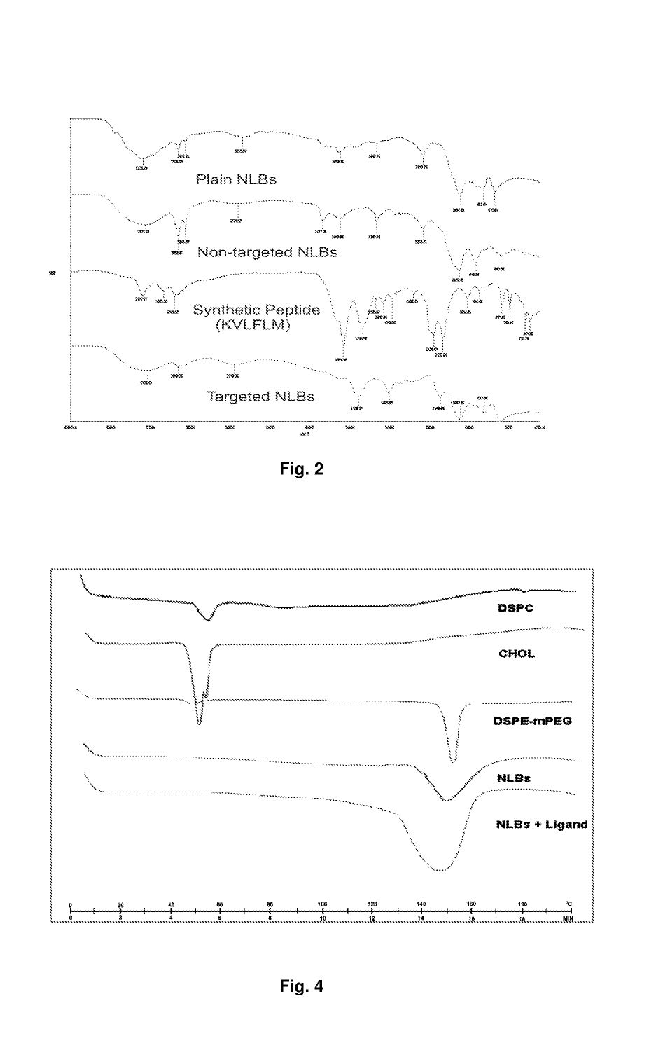

FTIR spectra is one of the most powerful chemical analytical techniques used for performing IR spectra, vibration, and characteristics of chemical functional group of phospholipids, liposome and synthetic peptide ligands (Weers and Sceuing, 1991). FTIR spectra were implemented to characterize the potential interactions in the non-targeted nano-lipobubbles and targeted nano-lipobubbles using a Nicolet Impact 400D FTIR spectrophotometer. FIG. 2 confirmed the molecular structural changes in the non-targeted nano-lipobubbles, and targeted nano-lipobubbles with the KVLFLM synthetic peptide (SEQ ID NO: 1). FIG. 3 confirmed the molecular structural changes in the non-targeted nano-lipobubbles and targeted nano-lipobubbles with the KVLFLS synthetic peptide (SEQ ID NO: 2). Overall, the results confirmed that there were interactions between the nano-lipobubbles and synthetic peptide ligands, and the formation of novel targeted nano-lipobubbles.

### Differential Scanning Calorimetry

A DSC study was carried out for DSPC, CHOL, DSPE-mPEG, non-targeted nano-lipobubbles and targeted nano-lipobubbles using a Mettler Toledo DSC instrument (DSC-823, Mettler Toledo, Switzerland). As illustrated in FIG. 4, DSPC, CHOL, DSPE-mPEG, non-targeted nano-lipobubbles and targeted nano-lipobubbles demonstrated different DSC thermograms during sample heating from 0 to 250° C. at a rate of 10° C./minute. The pretransition or endothermic transition peaks of pure DSPC, CHOL, and DSPE-mPEG were eradicated in non-targeted nano-lipobubbles, suggesting significant formation of nano-lipobubbles. An addition of 1 mole percent of synthetic peptide ligand broadened the endothermic transition peak of the non-targeted nano-lipobubbles. These results postulate interaction of the synthetic peptide ligand with the surface of the pegylated non-targeted nano-lipobubbles.

### Ex Vivo Cytotoxicity Studies of the Targeted Nano-Lipobubbles

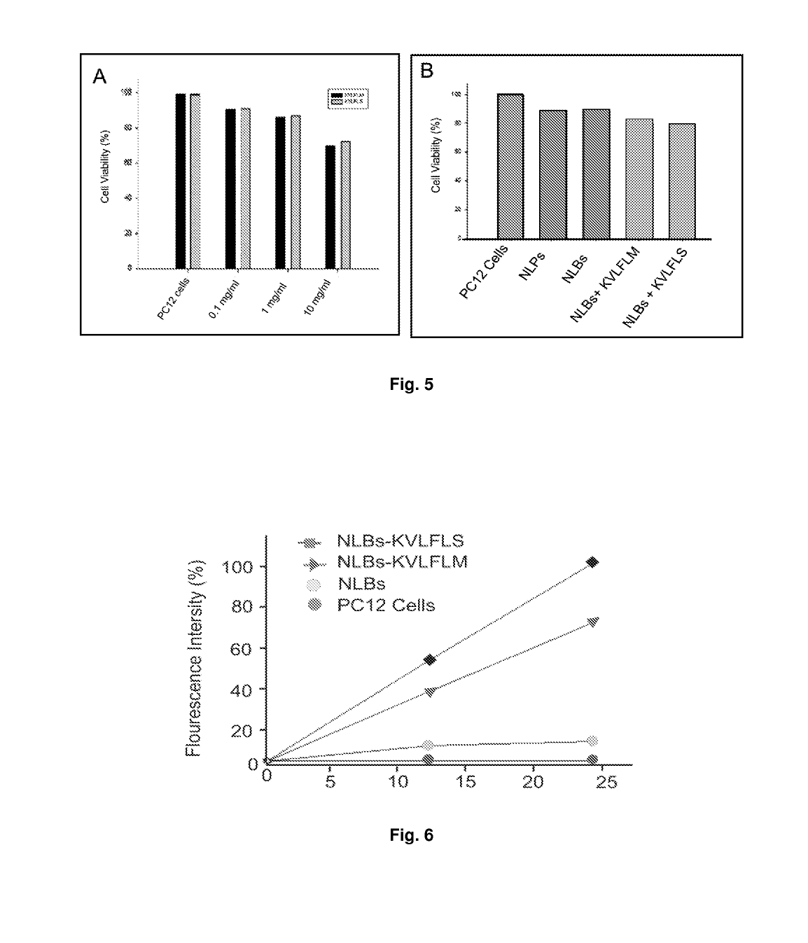

For cytotoxicity studies, targeted nano-lipobubbles, synthetic peptide ligands, non-targeted nano-lipobubbles and NLPs were assessed for their cytotoxic effects in the PC12 cell-line using the Victor X3 instrument. As illustrated in FIG. 5, the targeted nano-lipobubbles, synthetic peptides ligands alone, non-targeted nano-lipobubbles and NLPs demonstrated different cytotoxic effects on the PC12 cell-line after incubation for 24 hours. Although increased cell mortality was detected at different synthetic peptide concentrations of from 0.1 mg, 1 mg and 10 mg, low cell growth inhibition was exhibited when compared with the PC12 cell-line alone.

### Ex Vivo Uptake of the Targeted Nano-Lipobubbles

Rhodamine-labeled non-targeted nano-lipobubbles and targeted nano-lipobubbles with synthetic peptide ligand (KVLFLM (SEQ ID NO: 1) and KVLFLS (SEQ ID NO: 2)) were investigated for their uptake or delivery capability in the PC12 cell-line by the Victor X3 instrument. As shown in FIG. 6, fluorescence activity of targeted nano-lipobubbles at 0 hour, 12 hours and 24 hours was most efficiently detected in the PC12 cell-line. The non-targeted nano-lipobubbles showed the least fluorescence activity, suggesting that the enhanced cellular uptake of KVLFLM- and KVLFLS-targeted nano-lipobubbles was specifically mediated by the SEC-R receptor over-expressed on the surface of the PC12 cell-line with high uptake efficiency.

### Scaffold Morphology

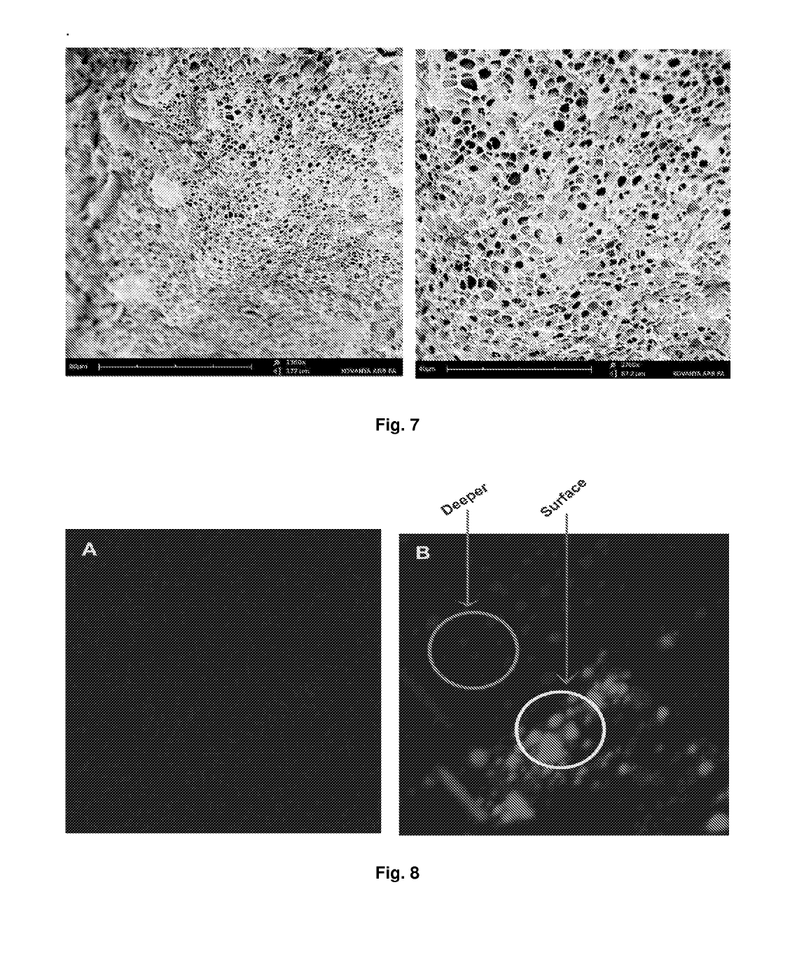

The surface morphology of the polymeric scaffold was examined by SEM. FIG. 7 shows the SEM micrograph of the chitosan/eudragit/sodium alginate porous scaffold at different magnifications (1360× and 2760×). The pores were relatively uniform in size and shape.

Confocal Microscopy to Confirm Nano-Lipobubble Encapsulation and Distribution within the Scaffold

To confirm nano-lipobubble encapsulation and distribution inside the porous scaffold, confocal microscopy was carried out. FIG. 8 shows strong fluorescence of rhodamine-labeled nano-lipobubbles inside the chitosan/eudragit RS-PO/sodium alginate porous scaffold. The CLSM micrograph shows rhodamine-labeled nano-lipobubble distribution all over the interior of the porous scaffold, toward the surface and deeper regions. No rhodamine fluorescence was observed in the scaffold only. These results confirm that the nano-lipobubbles were efficiently internalized into the porous scaffold. The fluorescence patterns postulate that the nano-lipobubbles were intact vesicles after incorporation within the porous scaffold.

### Example 2

### Drug Delivery Device for Treating Schizophrenia

### Materials and Methods

### Materials

Polymers utilized in this study include polyamide 6, 10 synthesized by a modified interfacial reaction. Hexamethylenediamine (Mw=116.2 g/mol), sebacoyl chloride (Mw=239.1 g/mol), anhydrous n-hexane, anhydrous potassium bromide, amitriptyline hydrochloride, and anhydrous sodium hydroxide pellets were used in the synthesis of polyamide 6, 10. The above-mentioned monomers, ethylcellulose, polycaprolactone, model drug chlorpromazine hydrochloride and cod-liver oil B.P. were purchased from Sigma Chemical Company (St Louis, Mo., USA). All other chemicals used were of analytical grade and commercially available.

### Preparation of Polymeric Implantable Membrane

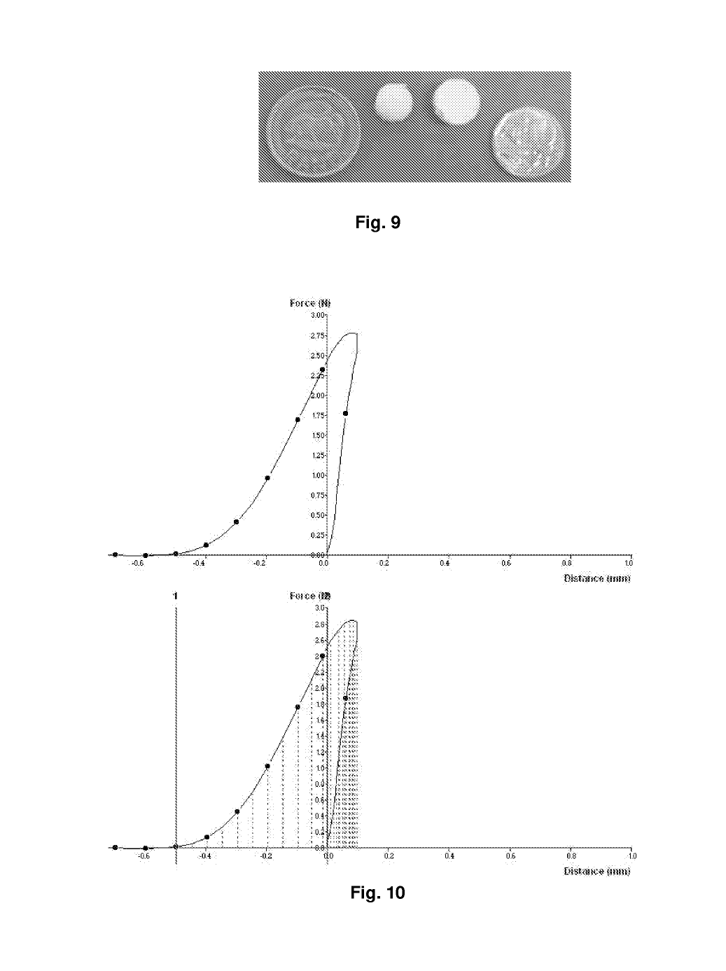

Polymeric membranes were prepared by a modified immersion precipitation reaction. 200 mg novel polyamide 6, 10 synthesized by modified interfacial polymerization reaction (Kolawole et al., 2007), was firstly dissolved in 2 ml formic acid. The solution was placed under magnetic stirring at 3000 rpm and the temperature was raised to 65° C. until all polyamide 6, 10 dissolved. Another solution comprising 200 mg ethylcellulose dissolved in 1 ml acetone was prepared. The polyamide-formic acid solution was then added to the ethylcellulose-acetone solution while under magnetic stirring at 3000 rpm. Stirring continued until the formation of a homogenous solution. The solution was left to stir for approximately an hour and upon completion of stirring, the solution was made to stand for approximately 10 minutes. 5 ml double de-ionized water was added to the solution. This resulted in the formation of a white gel-like precipitate at the interface. The precipitate was collected by filtration with a Buchner apparatus with the continuous addition of double de-ionized water. Following filtration, the precipitate was collected and appropriately moulded and left to dry under the fume hood for 24 hours. Resultant membranes were round, regular and exhibited surface porosity. Alternatively, the membranes can be frozen at −70° C. for 48 hours following moulding and then lyophilized for 48 hours producing highly porous scaffold-like membranous devices. FIG. 9 shows the size of devices formed according to this method, relative to South African five Rand (left) and 10 cent (right) coins.

### FTIR Spectrophotometric Analysis

Fourier transform infrared (FTIR) spectroscopy was undertaken on the resultant scaffolds to assess any structural variations in the polymeric membranous scaffold, as a result of any interactions in the formulation. A Spectrum 100 FTIR Spectrometer (PerkinElmer Life And Analytical Sciences Inc., Shelton, Conn. USA) was used to detect the vibration characteristics of chemical functional groups in a sample, in response to infrared light interactions.

### Morphological Characterization of Polymeric Membrane

Surface morphology was characterized by Scanning Electron Microscopy (SEM). Photomicrographs were taken at different magnifications and samples were prepared after sputter-coating with gold. Morphological characterization of the membrane revealed the shape, surface morphology and structure of the device.

### Determination of the Physicomechanical Properties of the Scaffolds

Textural analysis was used to determine the physicomechanical properties of the scaffolds in terms of its Brinell Hardness and deformation energy. The test was performed using the calibrated TA.XTplus Texture Analyzer (Stable Micro Systems, England) and is an indentation test where the scaffold was subjected to an abrupt impact that causes stress, and the hardness is determined by the volume of the indentation that was formed. The analyzer was fitted with a steel probe called the Brinell Hardness probe which causes the indentation in the scaffold causing the stress. The parameter settings employed for the analysis are outlined below in Table 1.

TABLE 1
Textural settings employed for determination of
BHN and deformation energy

| Parameters | Settings |
|---|---|
| Test mode | Compression |
| Pre-test speed | 1.00 mm/sec |
| Test speed | 0.5 mm/sec |
| Post-test peed | 2.00 mm/sec |
| Distance | 0.5 mm/sec |
| Target mode | Distance |

### Preparation of Cod-Liver Oil-Filled Nano-Liposhells

Chlorpromazine-loaded nano-liposhells were prepared by a modified melt-dispersion technique. 500 mg polycaprolactone was melted at 65° C. While in the molten state, 0.1 ml cod-liver oil B.P. was first added to the polycaprolactone. This was followed by the addition and dispersion of 50 mg chlorpromazine hydrochloride. Once adequately dispersed, the polycaprolactione-cod-liver oil-chlorpromazine dispersion was made to solidify and fuse by placing it under a fume hood. Once fused, the solid unit was granulated and then suspended in a polysorbate solution. This was followed by homogenization at 2000 rpm and ultrasonication at 80 Amp for 5 mins. Resultant nano-liposhells were frozen at −70° C. for 48 hours and thereafter lyophilized for 48 hours.

### Results and Discussion

### FTIR Spectrophotometric Analysis

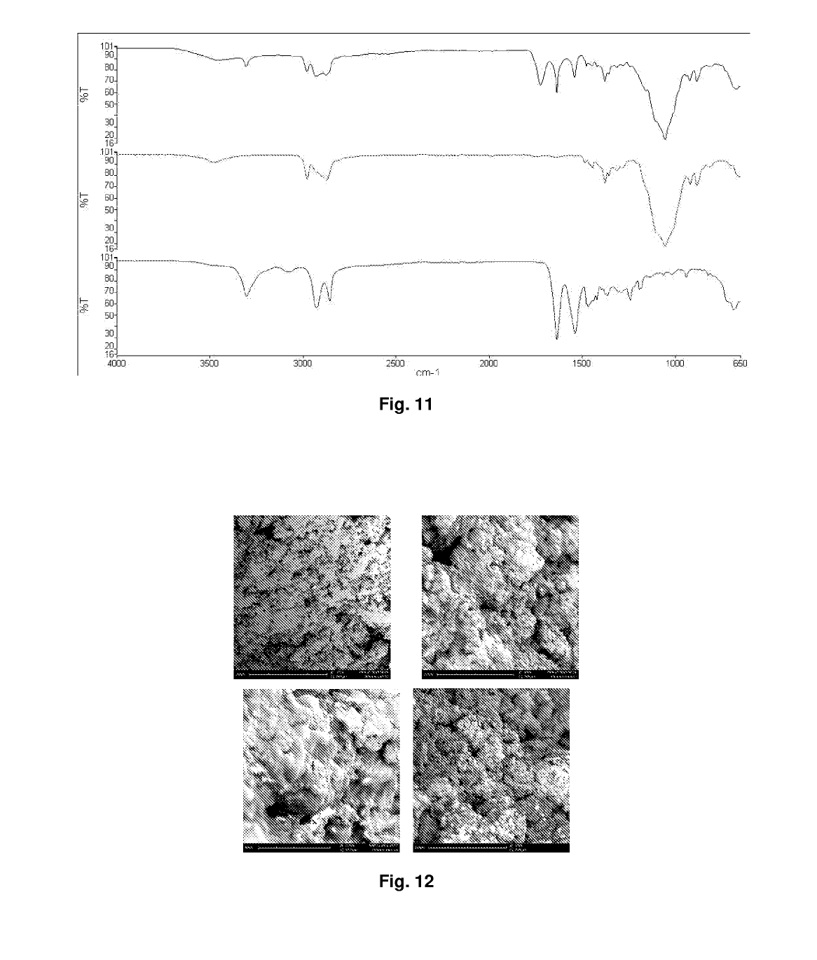

Structural characterization was performed on both the polyamide 6, 10, ethylcellulose and the polyamide-ethylcellulose membrane synthesized by modified immersion precipitation reaction. Results confirmed the presence and integrity of a combination of both polyamide and ethylcellulose functional groups in the membranes produced by modified immersion precipitation reaction (FIG. 11). This confirmed the formation of a novel polyamide-ethylcellulose implantable membrane device according to the invention.

### Morphological Characterization of Polymeric Membrane

FIG. 12 depicts SEM images of the novel polymeric membrane at varying magnifications. Membranes appear to be irregular and highly porous.

### Drug Entrapment Efficiency Testing

Drug-loading percentages of nano-liposhells were determined to assess the extent of drug entrapment during nano-liposhell formulation. Nano-liposhells were dissolved in PBS (pH 7.4) and assessed with ultraviolet spectrophotometry (Cecil CE 3021, Cecil Instruments Ltd., Milton, Cambridge, UK) against constructed standard curves.

$$D E E = \frac{Concentration of drug in nano \text{-} liposhells}{Theoretical concentration of drug} \times 100 \qquad Equation 1$$

The highest average drug entrapment efficiency (DEE) value of 40% was computed for chlorpromazine-loaded nano-liposhells with a 5:1 polymer:drug ratio. At lower polycaprolactone concentrations, DEE was significantly low. An average DEE value of 40% is satisfactory.

### Size Determination of Nano-Liposhells by Dynamic Light Scattering

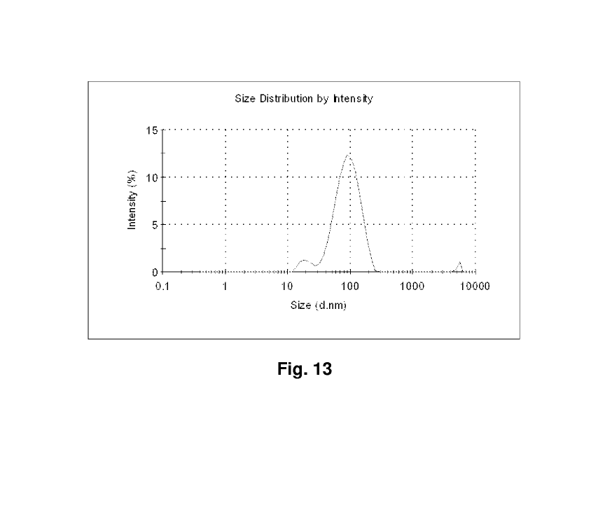

The Zetasizer NanoZS (Malvern Instruments Ltd, Malvern, Worcestershire, UK) incorporating dynamic light scattering techniques at 37° C. at varying angles was used to determine the average size and size distribution of the nano-liposhells produced, as well as their zeta potential and molecular weights. A nanoparticle z-average size of approximately 100 nm was recorded for preliminary chlorpromazine-free and chlorpromazine-loaded nano-liposhells (FIG. 13). This value is satisfactory as it is in the therapeutic size range for neuro-nanopharmaceutics.

### CONCLUSIONS

The polyamide-ethylcellulose scaffold was successfully synthesized and displayed no evidence of easy breakage. Resultant scaffolds were smooth, regular and of consistent size. Chlorpromazine-loaded nano-liopshells were also successfully prepared; however DEE values do appear to be somewhat low. Further research will be based on optimizing DEE and incorporating the drug-loaded nano-liposhells within the scaffold. Once this is complete, in vitro drug release testing will be performed to determine the extent and duration of drug release.

### REFERENCES

Arulmozhi D. K., Dwyer D. S. and Bodhankar S. L. Antipsychotic induced metabolic abnormalities: An interaction study with various PPAR modulators in mice, Life Sciences, 79(19), (2006), 1865-1872.
Awad A. G. and Voruganti L. N. P. The Burden of Schizophrenia on Caregivers: A Review, PharmacoEconomics, 26(2), (2008), 149-162.
Bodor N. and Buchwald P. Brain-Targeted Drug Delivery: Experiences to Date, American Journal of Drug Delivery, 1(1), (2003), 13-26.
Cavalieri, F., A. E. Hamassi, et al. 2006. “Tethering functional ligands onto shell of ultrasound active polymeric microbubbles.”Biomacromolecules 7 (2): 604-611.
Cheng Y. H., Illum L. and Davis S. S. Schizophrenia and Drug Delivery Systems, Journal of Drug Targeting, 8(2), (2000), pages 1075-117.
Chung, T. W., M. C. Yang, et al. (2006). “A fibrin encapsulated liposomes-in-chitosan matrix (FLCM) for delivering water-soluble drugs. Influences of the surface properties of liposomes and the crosslinked fibrin network.”International Journal of Pharmaceutics 311(1-2): 122-129.
Claas F. H. J. Drug-induced agranulocytosis: review of possible mechanisms and prospects for clozapine studies, Psychopharmacology, 99, (1989), S 113-S 117.
Dai, C., B. Wang, et al. (2006). “Preparation and characterization of liposomes-in-alginate (LIA) for protein delivery system.”Colloids and Surfaces B: Biointerfaces 47(2): 205-210.
Dhoot, N. O. and M. A. Wheatley. (2003). “Microencapsulated liposomes in controlled drug delivery: Strategies to modulate drug release and eliminate the burst effect.”Journal of Pharmaceutical Sciences 92: 679-689.
Ellis P. M. Clozapine: Fatal ‘constipation’ more common than fatal agranulocytosis, New Zealand Medicine and Medical Devices Safety Authority, 28(1), (2007), 7.
Frith C. Neuropsychology of schizophrenia: What are the implications of intellectual and experimental abnormalities for the neurobiology of schizophrenia? British Medical Bulletin, 52, (1996), 618-626.
Forssena, E and M. Willis. 1998. “Ligand-targeted liposomes.” Advanced Drug Delivery Reviews 29: 249-271.
Gharabawi G. M., Gearhart N. C., Lasser R. A., Mahmoud R. A., Zhu Y., Mannaert E., Naessens I., Bossie C. A., Kujawa M. and Simpson G. M. Maintenance therapy with once-monthly administration of long-acting injectable risperidone in patients with schizophrenia or schizoaffective disorder: a pilot study of an extended dosing interval, Annals of General Psychiatry, 6(3), (2007).
Gobin, A. S., R. Rhea, et al. (2006). “Silk-fibroin-coated liposomes for long-term and targeted drug delivery.”International Journal of Nanomedicine 1(1): 81-87.
Graff, A., M. Winterhalter, et al. (2001). “Nanoreactors from polymer-stabilized liposomes.”Langmuir 17: 919-923.
Greene, L. A. and Tischler, A. S. (1976). “Establishment of a noradrenergic clonal line of rat adrenal pheochromocytoma cells which respond to nerve growth factor.” Proceedings of the National Academy of Sciences 73:2424-2428.
Gutwinski S., Müller P. and Koller M. Intervals between hospitalisations in schizophrenia patients under antipsychotics in depot-form versus oral second generation antipsychotics, Psychiatr Prax, 34(6), (2007):289-291.
Hara, M and J. Miyake. 2001. “Calcium alginate gel entrapped liposomes.” Materials Science and Engineering 17: 101-105.
Hernot, S and A. L. Klibanov. 2008. “Microbubbles in ultrasound-triggered drug and gene delivery.” Advanced Drug Delivery Reviews 60: 1153-1166.
Hosny, K. M. (2010a). “Ciprofloxacin as Ocular Liposomal Hydrogel.”AAPS PharmSciTech 11(1): 241-246.
Hosny, K. M. (2010b). “Optimization of gatifloxacin liposomal hydrogel for enhanced transcorneal permeation.”AAPS PharmSciTech 20(1): 31-37.
Hynynen K. Macromolecular Delivery Across the Blood-Brain Barrier, Methods Mol Biol. 480, (2009), 175-185.
Immordino, M. L., F. Dosio, et al. (2006). “Stealth liposomes: review of the basic science, rationale, and clinical applications, existing and potential.”International Journal of Nanomedicine 1(3): 297-315.
Kane J. M., Aguglia E., Altamura A. C., Gutierrez J. L. A., Brunello N., Fleischhacker W. W., Gaebel W., Gerlach J., Guelfi J. D., Kissling W., Lapierre Y. D., Lindström E., Mendlewicz J., Racagni G., Carulla L. S. and Schooler N. R. Guidelines for depot antipsychotic treatment in schizophrenia, European Neuropsychopharmacoloay, 8(1), (1998), 55-66.
Kane J. M. Schizophrenia, The New England Journal of Medicine, 334, (1996), 34-42.
Klibanov, A. L. 1999. “Targeted delivery of gas-filled microspheres, contrast agents for ultrasound imaging.” Advanced Drug Delivery Reviews 37: 139-157.
Kolowale O. A., Pillay V. and Choonara Y. E. Novel Polyamide 6, 10 Variants Synthesized by Modified Interfacial Polymerization for Application as a Rate-Modulated Monolithic Drug Delivery System, Journal of bioactive and compatible polymers, 22, (2007), 281-313.
Krauze M. T., Forsayeth J., Park J. W., Bankiewicz K. S. Real-time Imaging and Quantification of Brain Delivery of Liposomes, Pharmaceutical Research, 23(11), (2006), 2493-2504.
Janssen, A. C. P. A., R. M. Schiffelers, et al. 2003. “Peptide-targeted PEG-liposome in anti-angiogenic therapy.”International Journal of Pharmaceutics 254 (1): 55-58.
Landgraf W., Li N. H., Benson J. R., New polymer enables near zero order release of drugs, Drug Delivery Technology, 5(2), (2005), 48-55.
Lieberman J. A., Stroup T. S., McEvoy J. P., Swartz M. S., Rosenheck R. A., Perkins D. O., Keefe R. S. E., Davis S. M., Davis C. E., Lebowitz B. D., Severe J. and Hsiao J. K. Effectiveness of Antipsychotic Drugs in Patients with Chronic Schizophrenia, The New England Journal of Medicine, 353, (2005), 1209-1223.
Lieberman J. A. Metabolic Changes Associated With Antipsychotic Use, J Clin Psychiatry, 6(2), (2004), 8-13.
Lincoln T. M., Lüllmann E. and Rief W. Correlates and Long-Term Consequences of Poor Insight in Patients with Schizophrenia. A Systematic Review, Schizophrenia Bulletin, 33(6), (2007), 1324-1342.
Machluf, M., O. Regev, et al. (1996). “Characterization of microencapsulated liposome systems for the controlled delivery of liposome associated macro molecules.”Journal of Controlled Release 43: 35-45.
Mak M., Fung L., Strasser J. F. and Saltzman W. M. Distribution of drugs following controlled delivery to the brain interstitium, Journal of Neuro-Oncology, 26, (1995), 91-10.
McCauley M. and Connolly G. Evidence for use of depot neuroleptic medication, Ir J Psych Med, 21(3), (2004), 95-99.
Meyenburg, S., H. Lilie, et al. (2000). “Fibrin encapsulated liposomes as protein delivery system. Studies on the in vitro release behavior.”Journal of Control Release 69(1): 159-168.
Michele, M., L. Fung, et al. (1995). “Distribution of drugs following controlled delivery to the brain interstitium.” Journal of Neuro-Oncology 26: 91-102.
Modi, G., V. Pillay, et al. (2009). “Nanotechnological applications for the treatment of neurodegenerative disorders.”Progress in Neurobiology 88: 272-285.
Mulik, R., V. Kulkarni, et al. (2009). “Chitosan-based thermosensitive hydrogel containing liposomes for sustained delivery of cytarabine.”Drug Development and Industrial Pharmacy 35(1): 49-56.
Nobs, L., F. Buchegger, et al. (2004). “Current Methods for Attaching Targeting Ligands to Liposomes and Nanoparticles.”Journal of Pharmaceutical Sciences 93(8): 1980-1992.
Pardridge W. M. The Blood-Brain Barrier: Bottleneck in Brain Drug Development, NeuroRX, 2(1), (2005), 3-14.
Pardridge W. M. Drug Delivery to the Brain, Journal of Cerebral Blood Flow & Metabolism, 17, (1997), 713-731.
Pavelic, Z., N. Skalko-Basnet, et al. (2005). “Development and in vitro evaluation of a liposomal vaginal delivery system for acyclovir. J 106 (2005) 34-43.”Journal of Controlled Release 106: 34-43.
Pfeiffer P N, Ganoczy D, Valenstein M. Dosing Frequency and Adherence to Antipsychotic Medications, Psychiatric Services, 59, (2008), 1207-1210.
Pranzatelli M. R. Innovations in drug delivery to the central nervous system, Drugs Today, 35(6), (1999), 435.
Rabin C., Liang Y., Ehrlichman R. S., Budhian A., Metzger K. L., Majewski-Tiedeken C., Winey K. I. and Siegel S. J. In vitro and in vivo demonstration of risperidone implants in mice, Schizophrenia Research, 98(1-3), (2008), 67-78.
Riche, E. L., B. W. Erickson, et al. (2004). “Novel long circulating liposome containing peptide library lipid conjugates: Synthesis and in vivo behavior.”Journal of Drug Targeting 12 (6): 355-361.
Shahiwala A. and Misra A. Pulmonary absorption of Liposomal Levonorgestrel, AAPS PharmSciTech, (2004).
Song, S., D. Liu, et al. (2008). “Peptide ligand mediated liposome distribution and targeting to EGFR expressing tumor in vivo.”International Journal of Pharmaceutics 363:155-161.
Stenekes, R. J., A. E. Loebis, et al. (2000). “Controlled release of liposomes from biodegradable dextran microspheres: a novel delivery concept.”Pharmaceutical Research 17(6): 690-695.
Suzuki, R., T. Takizawa, et al. (2007). “Gene delivery by combination of novel liposomal bubbles with perfluoropropane and ultrasound.” Journal of Control Release 117:130-136.
Torchilin, V. P. (2005). “Recent advances with liposomes as pharmaceutical carriers.” Nature Reviews Drug Discovery 4(2): 145-160.
Torchilin, V. P. (2008). “Tat peptide-mediated intracellular delivery of pharmaceutical nanocarriers.”Advanced Drug Delivery Reviews 60 548-558.
Unger E. C., Porter T., Culp W., Labell R., Matsunaga T. and Zutshi R. Therapeutic applications of lipid-coated microbubbles, Advanced Drug Delivery Reviews, 56(9), (2004), 1291-1314.
Verma, D. D., S. Verma, et al. (2003). “Particle size of liposomes influences dermal delivery of substances into skin.”International Journal of Pharmaceutics 258:141-151.
Wallace, D. G. and J. Rosenblatt (2003). “Collagen gel systems for sustained delivery and tissue engineering.” Advanced Drug Delivery Reviews 55(12): 1631-1649.
Weers, J. G and D. R. Scheuing. (1991). “Characterization of viscoelastic surfactant mixtures, I: Fourier transform infrared spectroscopic studies.”Colloids Surf. B: Biointerfaces 1 (55): 41-56.
Yagi N., Y. Ogawa et al. (2000). “Preparation of Functional Liposomes with Peptide Ligands and Their Binding to Cell Membranes.”Lipids 35: 673-679.
Ying, X., H. Wen, et al. (2010). “Dual-targeting dauorubicin liposomes improve the therapeutic efficacy of brain glioma in animals.”Journal of Controlled Release 141: 183-192.

1. An implantable intracranial device for the delivery of a pharmaceutically active agent to a human or animal for treating a mental or neurological disorder, the device comprising:
a pharmaceutically active agent for treating the disorder;
polymeric nano-lipoparticles into or onto which the pharmaceutically active agent is embedded, the nano-lipoparticles further comprising a peptide for targeting the nano-lipoparticles to a target molecule; and
a porous polymeric matrix incorporating the nanoparticles.

2. The device according to claim 1, wherein the mental or neurological disorder is Alzheimer's disease.

3. The device according to claim 1, wherein the mental or neurological disorder is schizophrenia.

4. The device according to claim 1, wherein the pharmaceutically active agent is a cholinesterase inhibitor, an NMDA receptor antagonist, an atypical antipsychotic or a typical antipsychotic.

5. The device according to claim 4, wherein the cholinesterase inhibitor is donepezil hydrochloride, rivastigmine or galantamine, or a salt thereof.

6. The device according to claim 4, wherein the NMDA receptor antagonist is memantine, or a salt thereof.

7. The device according to claim 4, wherein the typical antipsychotic is chlorpromazine, thioridazine, mesoridazine, levomepromazine, loxapine, molindone, perphenazine, thiothixene, trifluoperazine, haloperidol, fluphenazine, droperidol, zuclopenthixol or prochlorperazine, or a salt thereof.

8. The device according to claim 4, wherein the typical antipsychotic is chlorpromazine or a salt thereof.

9. The device according to claim 4, wherein the atypical antipsychotic is amisulpride, aripiprazole, asenapine, bifeprunox, blonanserin, clotiapine, clozapine, iloperidone, lurasidone, mosapramine, olanzapine, paliperidone, perospirone, pimavanserin, quetiapine, remoxipride, risperidone, sertindole, sulpiride, vabicaserin, ziprasidone or zotepine, or a salt thereof.

10. (canceled)

11. The device according to claim 1, wherein the nano-lipoparticles are formed from a composition comprising a polymer and the pharmaceutically active agent.

12. The device according to claim 11, wherein the composition additionally comprises at least one phospholipid.

13. The device according to claim 11, wherein the composition additionally comprises an essential fatty acid.

14. The device according to claim 13, wherein the nano-lipoparticles are formed from a composition comprising polycaprolactone, the pharmaceutically active agent and omega 3 fatty acids.

15. The device according to claim 12, wherein the nano-lipoparticles are formed from a composition comprising 1,2-distearoyl-sn-glycero-phosphatidylcholine (DSPC); cholesterol; 1,2-distearoyl-sn-glycero-3-phosphatidylcholinemethoxy(polyethyleneglycol)-2000] (DSPE-mPEG2000) conjugate and the pharmaceutically active agent.

16. The device according to claim 1, wherein the nano-lipoparticles are nano-liposhells.

17. The device according to claim 1, wherein the nano-lipoparticles are nano-lipobubbles.

18. (canceled)

19. The device according to claim 1, wherein the peptide ligand is conjugated to the nanoparticles.

20. The device according to claim 1, wherein the peptide ligand is capable of binding to the serpin-enzyme complex receptor (SEC receptor) in the brain.

21. The device according to claim 1, wherein the peptide ligand comprises an amino acid sequence selected from the group consisting of KVLFLM (SEQ ID NO: 1), KVLFLS (SEQ ID NO: 2) and KVLFLV (SEQ ID NO: 3).

22. The device according to claim 1, wherein the polymeric matrix is formed from a composition comprising ethylcellulose and modified polyamide 6, 10.

23. The device according to claim 1, wherein the polymeric matrix is formed from a composition comprising chitosan, eudragit and sodium alginate.

24. (canceled)

25. The device according to claim 1, which is implantable in the sub-arachnoid space.

26. The device according to claim 1, which is biodegradable.

27. The device according to claim 1, wherein:
the pharmaceutically active agent is an agent for treating Alzheimer's disease;
the polymeric nano-lipoparticles are nano-lipobubbles formed from 1,2-distearoyl-sn-glycero-phosphatidylcholine (DSPC); cholesterol; 1,2-distearoyl-sn-glycero-3-phosphatidylcholinemethoxy(polyethyleneglycol)-2000] (DSPE-mPEG2000) conjugate and the pharmaceutically active agent and are coupled to a peptide ligand having an amino acid sequence of KVLFLM (SEQ ID NO: 1), KVLFLS (SEQ ID NO: 2) or KVLFLV (SEQ ID NO: 3) targeting the serpin-enzyme complex receptor (SEC receptor) in the brain; and
the porous polymeric matrix is a porous scaffold formed from chitosan, eudragit and sodium alginate.

28. The device according to claim 1, wherein:
the pharmaceutically active agent is an antipsychotic agent for treating schizophrenia;
the polymeric nano-lipoparticles are nano-liposhells formed from polycaprolactone, the pharmaceutically active agent and omega 3 fatty acids; and
the porous polymeric matrix is formed from ethylcellulose and modified polyamide 6, 10.

29. A method of manufacturing an implantable intracranial device according to claim 1, the method comprising the steps of:
forming nano-lipoparticles containing a pharmaceutically active agent; and incorporating the nano-lipoparticles into a porous polymeric matrix.

30. A method of treating a mental or neurological disorder comprising implanting a device according to claim 1 into the cranium of a patient.
